(19) United States
(12) Patent Application Publication
(10) Pub. No.: US 2022/0257684 A1
(43) Pub. Date: Aug. 18, 2022

(54) ANTI-COCCIDIAL PHYTOGENIC FORMULATIONS

(71) Applicant: Academia Sinica, Taipei City (TW)

(72) Inventor: Wen-Chin YANG, Taichung County (TW)

(21) Appl. No.: 17/628,191

(22) PCT Filed: Jul. 18, 2020
(86) PCT No.: PCT/US20/42692

§ 371 Date: Jan. 18, 2022

Related U.S. Application Data
(60) Provisional application No. 62/876,633, filed on Jul. 20, 2019.

(51) Int. Cl. A61K 36/282; A61K 36/28; A61K 31/216; A61P 33/02; A61K 9/00; A23K 10/30; A23K 50/75

(52) U.S. Cl. CPC A61K 36/282; A61K 36/28; A61K 31/216; A23K 50/75; A61K 9/0056; A23K 10/30; A61P 33/02

(57) ABSTRACT

The present invention relates to anti-coccidial phytogenic formulations. Specifically, anti-coccidial compositions and use thereof are disclosed. An anti-coccidial composition comprises (a) an effective amount of Artemisia indica extract; and (b) an effective amount of Bidens pilosa extract. The composition may further comprise a pharmaceutically acceptable vehicle, excipient, or carrier, and/or an animal feed. The anti-coccidial compositions may be used in the manufacture of a medicament for killing coccidian oocysts, inhibiting oocyst sporulation, reducing sporozoite invasion, and lessening bloody stools, or for alleviating or treating coccidiosis, in a subject in need thereof. In another aspect, the invention relates to use of an anti-coccidial formulation comprising an effective amount of Artemisia indica extract, and a pharmaceutical acceptable vehicle in the manufacture of a medicament for killing coccidian oocysts and inhibiting coccidian oocyst sporulation, and sporozoite invasion.

### FIELD OF THE INVENTION

The present invention relates generally to phytogenic formulations for use in treatment of coccidiosis, and more specifically to anti-coccidial phytogenic formulations comprising a combination of Artemisia indica and Bidens Pilosa.

### BACKGROUND OF THE INVENTION

Coccidiosis is a major parasitic disease of poultry and causes a considerable economic loss in the poultry industry. The genus Eimeria, a coccidia subclass, belongs to spore-forming, unicellular protozoan parasites. They are intestinal parasites and can infect fish, reptiles, birds, mammals. Chickens are susceptible to at least 11 species of Eimeria, tenella, E. necatrix, E. brunetti and E. maxima are the more virulent species and E. acervulina, E. praecox and E. mitis are less virulent species in chickens. Eimeria infection is usually asymptomatic but shows severe clinical symptoms such as diarrhea, bloody droppings, dehydration, droopiness, listlessness, loss of appetite, paleness, ruffled feathers and huddling in young and immune-compromised animals.

The life cycle of Eimeria has intracellular, extracellular, asexual, and sexual stages. Once the chickens are infected with Eimeria, the parasites develop in the chicken and give rise to a microscopic egg (called an oocyst) which is passed out in the droppings. Under proper conditions of temperature and moisture the oocyst develops within one to two days to form a sporulated oocyst which is capable of infecting other chickens. At this stage the ooycyst contains eight bodies (called sporozoites), each of which is sable to enter cells in the chicken's intestine after the oocyst is eaten. After entering the cells, sporozoites divide many times producing offspring (merozoites). Each merozoite in turn may enter another intestinal cell. This cycle may repeat several times and cause a large number of intestinal cells being destroyed. Eventually, the cycle stops and sex cells (male and female) are produced. The male fertilizes the female to produce oocyst which ruptures from the intestinal cell and passes in the droppings. Thousands of oocysts may be passed in the droppings of an infected chicken. Therefore, poultry raised in crowded or unsanitary conditions are at great risk of becoming infected.

Plants are recognized as an excellent source for human and animal medicines. Bidens pilosa, an Asteraceae family, is claimed as an anti-coccidial herb for treating coccidiosis. U.S. Pat. No. 9,072,312 discloses Bidens Pilosa and polyacetylenic compounds for prevention and treatment of coccidiosis. Although Bidens Pilosa and its active compounds can prevent and treat coccidiosis, this prevention and treatment are sometimes ineffective in dirty chicken houses.

### SUMMARY OF THE INVENTION

In one aspect, the invention relates to an anti-coccidial composition comprising: (a) an effective amount of Artemisia indica extract; (b) an effective amount of Bidens pilosa extract, and (c) a pharmaceutically acceptable vehicle, excipient, or carrier.

In another aspect, the invention relates to an anti-coccidial composition comprising: (a) an effective amount of extracts or powders of Artemisia indica; and (b) an effective amount of extracts or powders of Bidens pilosa.

In one embodiment of the invention, the anti-coccidial composition comprises: (a) an effective amount of extracts of Artemisia indica; and (b) an effective amount of extracts of Bidens pilosa; or (a) an effective amount of extracts of Artemisia indica; and (b) an effective amount of powders of Bidens pilosa; or (a) an effective amount of powders of Artemisia indica; and (b) an effective amount of extracts of Bidens pilosa; or (a) an effective amount of powders of Artemisia indica; and (b) an effective amount of powders of Bidens pilosa. The anti-coccidial composition may further comprise a pharmaceutically acceptable vehicle, excipient, or carrier.

In one embodiment, the anti-coccidial composition is an oral dosage form. The oral dosage form may be selected from the group consisting of tablets, pills, softgel capsules, hard capsules, granules, powders, concentrates, liquids, molded balls and any combinations thereof.

In another embodiment, the anti-coccidial composition further comprises an animal feed, or a non-human animal feed or food.

In another embodiment, the animal feed is selected from the group consisting of poultry feed, fish food, amphibian feed, reptile food, bird feed, and non-human mammal food.

In another embodiment, the Artemisia indica extract and the Bidens pilosa extract are present in a ratio of about from 0.0001:1.0 to 1.0:1.0.

In another embodiment, the amount of Artemisia indica extract ranges from 0.0001% to 0.01% by weight of the composition. The amount of Artemisia indica may range from about 0.00005% to about 0.005% (w/w) or from about 0.001% to about 0.01% (w/w).

In another embodiment, the amount of Bidens pilosa extract ranges from 0.01% to 0.1% by weight of the composition. The amount of Bidens pilosa may range from about 0.005% to about 0.05%.

In another embodiment, the Artemisia indica extract comprises chlorogenic acid.

The invention also relates an anti-coccidial composition consisting essentially of or consisting of: (a) an effective amount of extracts or powders of Artemisia indica; (b) an effective amount of extracts or powders of Bidens pilosa, and (c) a non-human animal feed or food.

The invention further relates to an anti-coccidial composition consisting essentially of or consisting of: (a) an effective amount of extracts or powders of Artemisia indica; and (b) an effective amount of extracts or powders of Bidens pilosa.

In one embodiment of the invention, the anti-coccidial composition is in a non-aqueous liquid, aqueous liquid, suspension or powder form.

Further in another aspect, the invention relates to use of the anti-coccidial composition of the invention in the manufacture of a medicament for killing coccidian oocysts and inhibiting coccidian oocyst sporulation.

Further in another aspect, the invention relates to use of the anti-coccidial composition of the invention in the manufacture of a medicament for killing coccidian oocysts, inhibiting oocyst sporulation, reducing sporozoite invasion, and lessening bloody stools, or for alleviating or treating coccidiosis, in a subject in need thereof.

Yet in another aspect, the invention relates to use of an anti-coccidial formulation comprising an effective amount of Artemisia indica extract, and a pharmaceutical acceptable vehicle in the manufacture of a medicament for killing coccidian oocysts and inhibiting coccidian oocyst sporulation, and sporozoite invasion.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIGS. 1A-C are photomicrographs (upper panels) and bar graphs (lower panels) illustrating the in vitro effects of formulations comprising A. indica (AI), B. pilosa (BP), or a mixture of A. indica (AI) and B. pilosa (BP) on viability of Eimeria. tenella (ET) oocysts. The oocysts were pre-treated with PBS (negative control, NC), boiling (positive control, PC) or plant extracts containing AI (1A), BP (1B) or both AI and BP (1C) at the indicated dosages for 30 min. After propidium iodide (PI) staining, the oocysts viability was examined using a fluorescent microscope (left bottom panels) or a light microscope (left top panels). Bright field showed the number of the oocysts. Percentage of PI-positive oocysts, presented as mean±SE, was plotted into bar graphs (right panels); scale bar is 100 μm.

FIGS. 2A-C are photomicrographs (upper panels) and bar graphs (lower panels) illustrating the in vitro effects of formulations comprising A. indica (AI), B. pilosa (BP), or a mixture of A. indica (AI) and B. pilosa (BP) on viability of E. tenella (ET) oocysts and its sporulation. The oocysts were pre-treated with PBS, sodium dithionite (SD), an inhibitor of oocyst sporulation or plant extracts containing AI (2A), BP (2B), or both AI and BP (2C) at the indicated dosages for 48 h. The oocysts were induced to sporulate by potassium dichromate for 2 days. The percentage of sporulating oocysts was counted using a microscope (left panels) and plotted into bar graphs (right panel); scale bar is 200 μm. Arrow indicates sporulating oocysts whilst arrowhead indicates non-sporulating oocysts.

FIGS. 3A-C are photomicrographs (left panels) and bar graphs (right panels) illustrating the in vitro effects of formulations composed of A. indica (AI), B. pilosa (BP), or a mixture of A. indica (AI) and B. pilosa (BP) on E. tenella (ET) sporozoite invasion. Madin-Darby bovine kidney (MDBK) cells were incubated with salinomycin (Sal), or plant extracts containing A. indica (AI)(3A), B. pilosa (BP) (3B) or a mixture of AI and BP (3C) at indicated dosages for 0.5 h. The sporozoites were added to the MDBK cells for additional 2 h. After washing, the MDBK cells were stained with hematoxylin and eosin (H&E) and counted. The invasion percentage (%) was plotted into bar graphs. The inset represents a magnified area. Scale bars of microscope in 100× and 40× (inset) are 10 μm and 50 μm (inset), respectively. Arrowhead indicates the MDBK cells infected with sporozoites.

FIG. 4A is a schematic drawing showing the experimental protocol used in in vivo studies. Birds in untreated groups had daily access to standard diet, and birds in treated groups were fed with diet containing salinomycin, A. indica (AI), B. pilosa (BP), or a mixture of AI plus BP. On day 7, chickens were challenged with PBS or E. tenella (ET) sporulated oocysts (1×10⁴) by gavage. The survival rate in each group was monitored from day 1 to 7 post infection.

FIG. 4B is a graph showing survival rate in each group of FIG. 4A. Chickens were divided into eight groups: Group 1 (G1), uninfected and fed with a standard diet; Group 2 (G2), ET-infected and fed with a standard diet; Group 3 (G3) to Group 8 (G8), ET-infected and fed with diet containing salinomycin, BP alone, a combination of BP and different dosages of AI, or AI alone without BP as indicated. Each group has 15 chicks and P values are indicated.

FIG. 5 is a bar graph showing percentage of bloody stools in each chicken group of FIG. 4A-B on Day 7 post E. tenella (ET) infection. Chicken stools were classified on Day 1 to Day 7 post infection.

### DETAILED DESCRIPTION OF THE INVENTION

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

By about 0.00005% to about 0.005% it meant that all hundred-thousandth, ten-thousandth, thousandth, hundredth, tenth and integer unit amounts within the range are specifically disclosed as part of the invention. Thus, 0.00005%, 0.00006%, 0.00007% . . . 0.00009%, 0.0001%, 0.0002% . . . and 0.0009%, 0.001%, 0.0011% . . . and 0.0048, 0.0049, 0.005% unit amounts are included as embodiments of this invention.

By about 0.0001% to about 0.01% it meant that all ten-thousandth, thousandth, hundredth, tenth and integer unit amounts within the range are specifically disclosed as part of the invention. Thus, 0.0001%, 0.0002%, 0.0003% . . . 0.0009%, 0.001%, 0.0011% . . . and 0.0098, 0.0099, 0.01% unit amounts are included as embodiments of this invention.

By about 0.005% to about 0.05% it meant that all thousandth, hundredth, tenth and integer unit amounts within the range are specifically disclosed as part of the invention. Thus, 0.005%, 0.006%, 0.007% . . . 0.0099, 0.01%, 0.011% . . . and 0.048%, 0.049%/a, 0.05% unit amounts are included as embodiments of this invention.

By about 0.01% to about 0.1% it meant that all hundredth, tenth and integer unit amounts within the range are specifically disclosed as part of the invention. Thus, 0.01%, 0.02%, 0.03% . . . and 0.08%, 0.09%, 0.1% unit amounts are included as embodiments of this invention.

The term “a mixture” shall generally mean any combination or blend of different kinds.

The invention relates to the anti-coccidial activity of phytogenic formulations comprising Bidens

Preparation of B. pilosa and Artemisia indica Extracts

Artemisia indica extract. Artemisia indica plants were collected from the campus of Miaoli, Taiwan. Approximately 1 kg of dried whole plants in their entirety was refluxed in 10 L of 100° C. water for two hours. After removal of aqueous phase, insoluble materials was again refluxed in 10 L of water for two hours. The combined aqueous solutions were lyophilized to yield crude extract (0.15 kg). The chlorogenic acid was used as a marker of batch consistency. Chlorogenic acid was obtained and characterized by ¹H NMR and ¹³C NMR.

Bidens pilosa extract. B. pilosa plants were collected and processed using a similar method as described above. Approximately 1 kg of dried whole plants was refluxed in 10 L of 100° C. water for two hours and the extraction was repeated once. The combined water solutions were lyophilized to yield crude extract (0.12 kg). The cytopiloyne was used as a marker of batch consistency. Polyacetylenic compounds was obtained and characterized by ¹H NMR and ¹³C NMR. The pure compounds thus obtained can be further derivatized to provide a number of other polyacetylenic compounds (U.S. Pat. No. 7,763,285, and Kusano et al (JP 2004083463), all of which are incorporated herein by reference in their entireties).

The extract of A. indica and B. pilosa are used in form of powder. Calculation of the percentage of the plant extract powder is as follows: the plant powder weight/the plant powder weight±basic chicken feed=% of the plant powder.

The invention relates to the discovery of anti-coccidial properties of Artemisia indica and a formulation comprising Artemisia indica and Bidens pilosa.

### Examples

### Materials and Methods

Chemicals. Salinomycin, PBS, and H&E stain were purchased from SIGMA-ALDRICH™. Artemisia indica and B. pilosa were extracted in boiling water and mixed at a variety of ratios for further use.

Invasion assay. Madin-Darby bovine kidney (MDBK, ATCC® CCL-22) cells, grown in DMEM containing fetal bovine serum (10%) and supplements, were seeded onto glass cover slips in 24 wells at 2×10⁵ cells/well. One day later, the cells were pre-incubated with DMEM medium containing salinomycin, or plant extracts and phytochemicals at doses indicated for 0.5 h. Fresh sporozoites (2×10⁵) were added to the cells for additional 4 h. Ater extensive PBS washing, the cells were fixed and stained with H&E. Photographs were taken with a microscope. Invasion percentage (%) was obtained by the formula, 100%×(the number of cells invaded by sporozoites/total cell number).

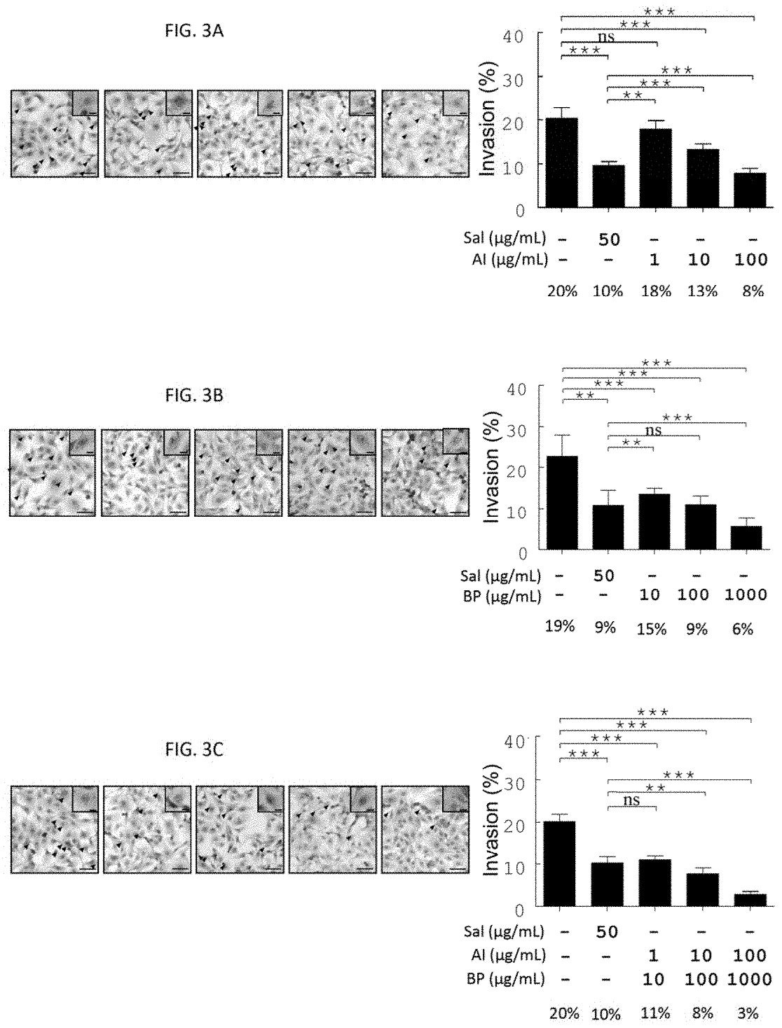

Viability test. The E. tenella sporozoites were incubated with plant extract or salinomycin for 4.5 h. Microscopy was used to distinguish life and death in sporozoites (FIG. 3). Survival rate (%) was obtained by normalization of the dead cell number by total cell number multiplied by 100%.

Sporulation assay. The E tenella oocysts were pre-treated with PBS, boiling (100° C. for 30 min) or plant extracts at indicated doses for 48 h. The oocysts were incubated with 2% potassium dichromate for 2 days before sporulation. The percentage of sporulating oocysts (%) was counted.

Propidium iodide (PI) staining of E. tenella sporozoites. The ET oocysts underwent PBS (1 h), boiling treatment (100° C. for 30 min) or incubation with plant extracts at indicated doses for 1 h. The oocysts were stained with PI. After PBS washing, the oocysts were examined using a microscope.

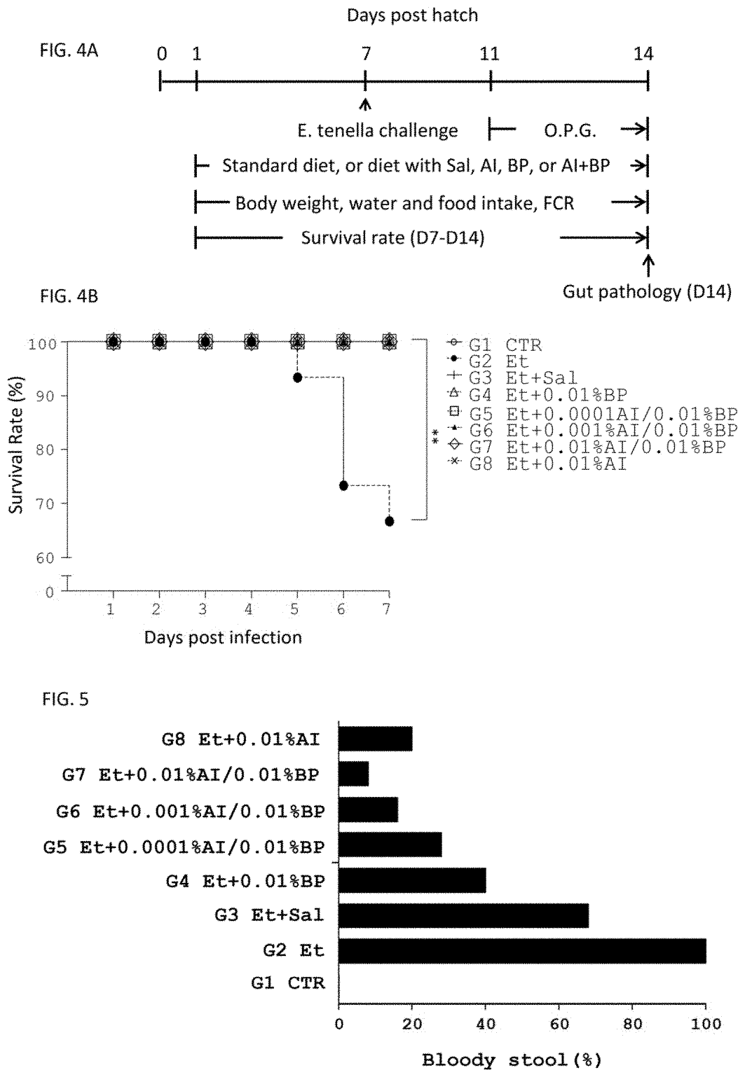

Birds, diets, and experimental design. One-day-old Lohmann broiler chicks hatched at Taichung Hatchery (Taiwan) were wing-banded upon arrival, weighed, and randomly placed in Petersime starter brooder units. At 1 to 2 days post-hatching, the birds were given free access to water and diets. The diets were formulated by mixing a base diet with a carrier alone (control diet) or with an indicated dose of a phytogenic formulation comprising Artemisia indica (AI), B. pilosa (BP), or both AI and BP. After grouping, birds were tube-fed with Eimeria oocysts to get infection. Growth performance (body weight and feed conversion), pathology (bloody droppings and gut morphometry) and survival rate were measured and examined (FIG. 4A). All birds were maintained in the institutional animal facility and handled according to institutional guidelines.

Immunohistochemical staining. Multiple parallel sections of the ceca from chickens that had access to control diet and the diet containing Artemisia indica (AI), B. pilosa (BP), or both for 14 days were flash-frozen. The sections were stained with H&E or anti-insulin antibody, with development of diaminobenzidine tetrahydrochloride, followed by image analysis (Yang et al. Research in Veterinary Science (2015) 98:74-81).

Statistical analysis. The results from three or more independent experiments were presented as mean±S.E. Data were analyzed by ANOVA. Differences with P value less than 0.05 were considered statistically significant.

### Results

### Effects of Plant Extracts on E. Tenella Oocyst Survival, Oocyst Sporulation and Sporozoite Invasion

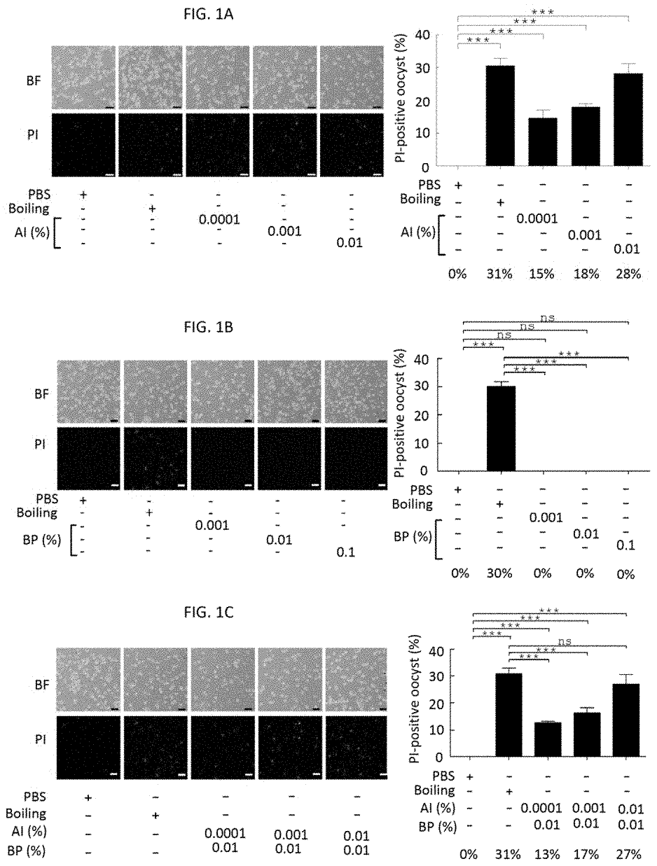

To investigate the effects of the extracts of A. indica (AI), B. pilosa (BP) and combined extracts of AI and BP, the direct killing activities of AI, BP and a combination of AI and BP in E. tenella (ET) oocysts were examined, respectively. Boiling treatment as a positive control effectively killed 30% of ET oocysts as demonstrated by propidium iodide (PI) staining (Boiling, FIG. 1A). AI directly killed ET oocysts in a dose-dependent manner (FIG. 1A, right panel). BP failed to kill the oocysts (FIG. 1B). A combination of AI and BP killed ET oocysts (FIG. 1C).

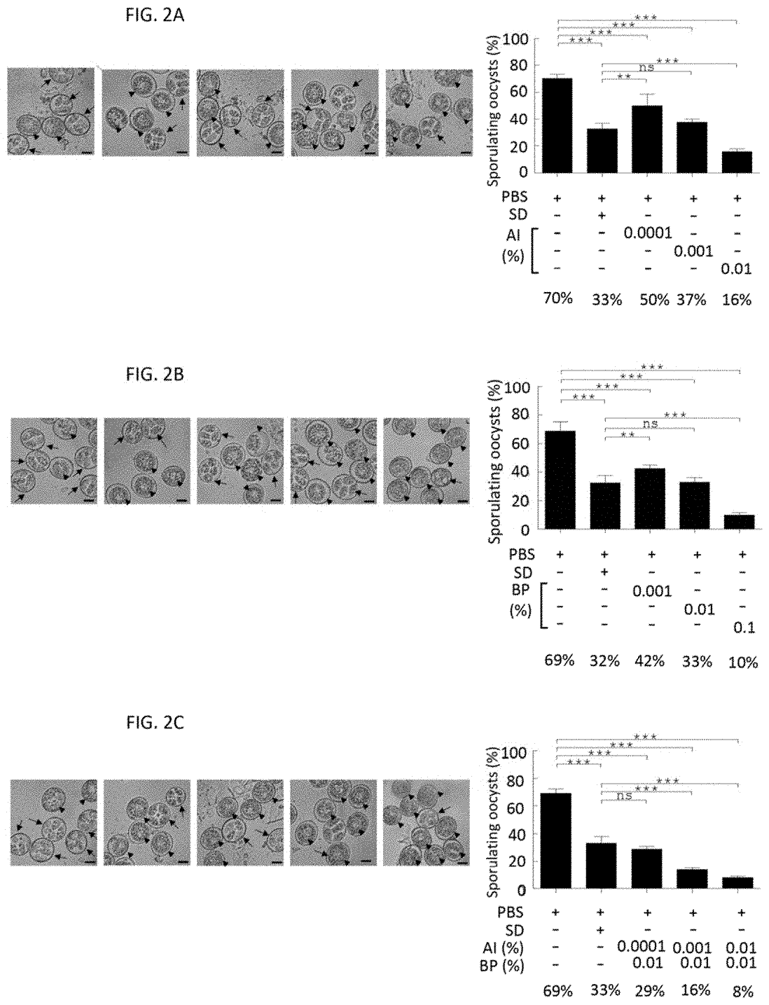

The effects of the plant extracts on sporulation of E. ET oocysts were tested. Seventy percent of ET oocysts were able to sporulate in the in vitro culture (PBS, FIG. 2A). Treatment with sodium dithionite (SD) as a positive control reduced sporulation of E. ET oocysts by 40% as compared to the PBS treatment group (SD, FIG. 2A, right panel). A. indica (AI) reduced E. ET oocyst sporulation in a dose-dependent manner (FIG. 2A). B. pilosa (BP) also dose-dependently inhibited sporulation of ET oocysts (FIG. 2B). A combination of AI and BP inhibited sporulation of ET oocysts in a synergistic manner (FIG. 2C).

The effect of B. pilosa on the entry of ET sporozoites into MDBK cells was examined. As reported by Yang et al. (Scientific Reports (2019) 9:2896), ET sporozoites could invade into 20% of the cells (FIG. 3A, right panel). Salinomycin, an anti-coccidial drug, at 50 μg/ml, decreased the ET sporozoite invasion to 10%. A. indica (AI) dose-dependently reduced ET sporozoite invasion (FIG. 3A, right panel). B. pilosa (BP) also reduced the invasion of ET sporozoites into MDBK cells in a dose-dependent manner (FIG. 3B, right panel). A combination of AI and BP decreased ET sporozoite invasion in a synergistic manner (FIG. 3C, right panel). The data demonstrate that treatment with a combination of AI and BP could interfere with the life cycle of ET at the oocysts, oocyst sporulation, and sporozoite invasion stages of ET. A combination of AI and BP were more potent in inhibiting ET oocyst sporulation and ET sporozoite invasion than AI or BP alone. The data suggest AI and BP have different anti-coccidial modes of mechanism.

Effects of Plant Extracts on Survival Rate, Body Weight Gain, and Feed Conversion Rate in E. tenella Challenged Chickens

The anti-coccidial effects of the extracts of A. indica (AI), B. pilosa (BP) and a combination of both extracts were examined in vivo. Chickens were randomly divided into 8 groups and given daily (Day 1 to Day 14) access to standard chicken feed (Groups 1 and 2), feed containing salinomycin (Group 3), or feed containing the plant extracts (Groups 4 to 8) at the indicated doses (FIG. 4A).

FIG. 4B shows the survival rate of chickens having access to standard feed dropped from 100% (CTR, Group 1) to 60% (Et, Group 2) after ET infection. The survival rate of the infected chickens having access to feed containing salinomycin was 100% (Group 3). Similarly, the survival rate of the infected chickens having access to feed containing AI, BP, or a combination of AI and BP were all 100% (Groups 4-8).

Table 1 shows the effect of plant extracts on the body weight gain in the ET challenged chickens. Body weights (BW) of the chickens from FIG. 4B were monitored on Days 1, 7 and 14, and body-weight (BW) gains were calculated on Days 7 and 14. On Day 7 prior to ET infection, chickens among different groups had similar body weight gain (BW on Day 7 minus BW on Day 1). Chickens in Groups 2 to 8 (G2 to G8) were challenged with ET oocysts (1×10⁴), and chickens in the control group (G1) challenged with PBS. On Day 14 post FT infection, the average body weight gain (BW on Day 14 minus BW on Day 1) of the uninfected unmedicated chickens (Group 1), infected unmedicated chickens (Group 2), infected chickens medicated with salinomycin (Group 3), 0.01% BP (Group 4), 0.0001% AI plus 0.01% BP (Group 5), 0.0010% AI plus 0.01% BP (Group 6), 0.01% AI plus 0.01% BP (Group 7), and 0.01% AI (Group 8) were 88.84 g, 50.80 g, 56.33 g, 61.98 g, 62.91 g, 64.65 g, 67.66 g, and 59.78 g, respectively. The data suggest that a combination of AI and BP had a better body weight gain than either one alone.

TABLE 1

| | BWG (g)^{a} | P value^{b} | P value^{c} | BWG (g)^{a} | P value^{b} | P value^{c} |
|---|---|---|---|---|---|---|
| Group | Day 7-1 | Day 7-1 | Day 7-1 | Day 14-1 | Day 14-1 | Day 14-1 |
| 1 (n = 15, 0) | 28.91 ± 1.45 | | | 88.84 ± 1.95 | | |
| 2 (n = 15, 1 × 10⁴) | 28.55 ± 1.04 | >0.05 | | 50.80 ± 2.13 | <0.001 | |
| 3 (n = 15, 1 × 10⁴) | 27.64 ± 1.16 | >0.05 | >0.05 | 56.33 ± 3.15 | <0.001 | <0.01 |
| 4 (n = 15, 1 × 10⁴) | 27.86 ± 1.57 | >0.05 | >0.05 | 61.98 ± 1.86 | <0.001 | <0.001 |
| 5 (n = 15, 1 × 10⁴) | 28.96 ± 1.70 | >0.05 | >0.05 | 62.91 ± 2.83 | <0.001 | <0.05 |
| 6 (n = 15, 1 × 10⁴) | 29.31 ± 1.77 | >0.05 | >0.05 | 64.65 ± 2.39 | <0.001 | <0.01 |
| 7 (n = 15, 1 × 10⁴) | 29.99 ± 0.66 | >0.05 | >0.05 | 67.66 ± 1.96 | <0.001 | <0.001 |
| 8 (n = 15, 1 × 10⁴) | 29.17 ± 1.93 | >0.05 | >0.05 | 59.78 ± 2.14 | <0.001 | <0.05 |

^{a}Body weight gain (BWG) was obtained by the formula: body weight on Day 7 or Day 14 minus body weight on Day 1. Day 7-1 stands for Day 7 BW gain, and Day 14-1 stands for Day 14 BW gain,
^{b}Statistic comparison made between Group 1 and other groups.
^{c}Statistic comparison made between Group 2 and other groups.

Table 2 shows the effect of plant extracts on feed conversion rate (FCR) in ET challenged chickens. On Day 14 post ET infection, the average FCR of control chickens (Group 1), infected chickens (Group 2), infected chickens fed with salinomycin (Group 3), 0.01% BP (Group 4), 0.0001% AI plus 0.01% BP (Group 5), 0.001% AI plus 0.01% BP (Group 6) 0.01% AI plus 0.01% BP (Group 7) and 0.01 AI (Group 8) were 2.24, 3.38, 3.05, 2.60, 3.01, 3.04, 2.88 and 2.96, respectively. The results indicate that AI, BP, and a combination of AI and BP all significantly ameliorated the reduction in the body weight gain and also ameliorated the increase in FCR caused by the ET infection to a greater degree than salinomycin or control feed alone. Part of this amelioration could be attributed to the weight-gaining effect of the plant extracts.

TABLE 2

| | FCR | | | | | |
|---|---|---|---|---|---|---|
| | FCR | P value^{a} | P value^{b} | FCR | P value^{a} | P value^{b} |
| Group | Day 7-1 | Day 7-1 | Day 7-1 | Day 14-1 | Day 14-1 | Day 14-1 |
| 1 (n = 15, 0) | 2.10 ± 0.09 | | | 2.24 ± 0.05 | | |
| 2 (n = 15, 1 × 10⁴) | 2.20 ± 0.06 | >0.05 | | 3.38 ± 0.13 | <0.001 | |
| 3 (n = 15, 1 × 10⁴) | 2.42 ± 0.08 | >0.05 | >0.01 | 3.05 ± 0.13 | <0.001 | >0.05 |
| 4 (n = 15, 1 × 10⁴) | 2.17 ± 0.08 | >0.05 | >0.05 | 2.60 ± 0.09 | <0.001 | <0.001 |
| 5 (n = 15, 1 × 10⁴) | 2.27 ± 0.12 | >0.05 | >0.05 | 3.01 ± 0.12 | <0.001 | >0.05 |
| 6 (n = 15, 1 × 10⁴) | 2.46 ± 0.14 | >0.05 | >0.05 | 3.04 ± 0.11 | <0.001 | >0.05 |
| 7 (n = 15, 1 × 10⁴) | 2.16 ± 0.05 | >0.05 | >0.05 | 2.88 ± 0.10 | <0.001 | <0.01 |
| 8 (n = 15, 1 × 10⁴) | 2.51 ± 0.17 | >0.05 | >0.05 | 2.96 ± 0.15 | <0.001 | >0.05 |

Feed conversion rate (FCR) in different, group of chickens was obtained by the formula: feed intake (Kg) divided by body weight gain on day 7 or 14. Day 7-1 stands for Day 7 BW gain, and Day 14-1 stands for Day 14 BW gain.
^{a}Statistic comparison made between Group 1 and other groups.
^{b}Statistic comparison made between Group 2 and other groups.

Effect of Plant Extracts on Fecal Oocyst Excretion in E. tenella Challenged Chickens

Table 3 shows fecal oocyst excretion in each group. Excretion of Eimeria oocysts in the feces of ET-infected chickens is an indicator of Eimeria multiplication. The oocysts per gram feces (OPG) in the chickens from FIG. 4B were counted from Day 3 to Day 7 post ET infection. No oocysts in the feces were detected in the uninfected unmedicated control group (Group 1, Table 3). Fecal oocyst excretion was first detected on Day 4 post infection in all the ET-infected groups and reached its peak on Day 7 post infection (Group 2). The infected birds fed with salinomycin (Group 3) had slightly less oocysts per gram of feces than the infected unmedicated birds (Group 2). The infected birds fed with 0.01% BP, 0.0001% AI plus 0.01% BP, 0.001% AI plus 0.01% BP, 0.01% AI plus 0.01% BP, and 0.01% AI (Groups 4 to 8), respectively, had significantly less oocysts per gram of feces than the infected unmedicated birds (Group 2). The data suggest that a combination of AI and BP reduced more OPG than either one alone.

TABLE 3

| | Days post-infection | | | | |
|---|---|---|---|---|---|
| | 3 | 4 | 5 | 6 | 7 |
| Group | Ln(OPG ± 1) | Ln(OPG ± 1) | Ln(OPG ± 1) | Ln(OPG ± 1) | Ln(OPG ± 1) |
| 1 (n = 15, 0) | 0 ± 0 | 0 ± 0 | 0 ± 0 | 0 ± 0 | 0 ± 0 |
| 2 (n = 15, 1 × 10⁴) | 0 ± 0 | 3.86 ± 0.04^{a} | 4.95 ± 0.02^{a} | 5.31 ± 0.06^{a} | 5.56 ± 0.02^{a} |
| 3 (n = 15, 1 × 10⁴) | 0 ± 0 | 3.45 ± 0.16^{a,b} | 4.49 ± 0.02^{a,b} | 4.89 ± 0.01^{a,b} | 5.33 ± 0.01^{a,b} |
| 4 (n = 15, 1 × 10⁴) | 0 ± 0 | 3.26 ± 0.15^{a,b} | 4.42 ± 0.05^{a,b} | 4.86 ± 0.02^{a,b} | 5.18 ± 0.02^{a,b} |
| 5 (n = 15, 1 × 10⁴) | 0 ± 0 | 3.26 ± 0.15^{a,b} | 4.42 ± 0.01^{a,b} | 4.87 ± 0.03^{a,b} | 5.17 ± 0.01^{a,b} |
| 6 (n = 15, 1 × 10⁴) | 0 ± 0 | 3.20 ± 0.10^{a,b} | 4.41 ± 0.03^{a,b} | 4.84 ± 0.03^{a,b} | 5.15 ± 0.01^{a,b} |
| 7 (n = 15, 1 × 10⁴) | 0 ± 0 | 3.15 ± 0.28^{a,b} | 4.39 ± 0.02^{a,b} | 4.77 ± 0.02^{a,b} | 5.11 ± 0.01^{a,b} |
| 8 (n = 15, 1 × 10⁴) | 0 ± 0 | 3.30 ± 0.17^{a,b} | 4.39 ± 0.02^{a,b} | 4.88 ± 0.03^{a,b} | 5.23 ± 0.01^{a,b} |

The OPG values of the chickens in each group were transformed into ln(x ± 1) and analyzed with ANOVA using the GEM procedure of SAS system under a normal distribution. The number (n) of chickens in each group is indicated.
^{a}Statistic comparison made between Group 1 and other groups.
^{b}Statistic comparison made between Group 2 and other groups.

Effect of Plant Extracts on Intestinal Lesions in E. tenella Challenged Chickens

Table 4 shows gross cecal lesion scores on Day 7 post ET infection. Gross lesions in the ceca of the chickens from FIG. 4B were examined and scored. The uninfected unmedicated control chickens (Group 1, Table 4) had no lesions in the ceca (score=0). The ET infection caused more gross cecal lesions in the gut of unmedicated chickens, as evidenced by an average lesion score of 4 (Group 2) on Day 7 post infection. The infected birds fed with salinomycin had an average lesion score of 3.86 (Group 3). The infected birds fed with 0.01% BP, 0.0001% A plus 0.01% BP, 0.001% AI plus 0.01% BP, 0.01% AI plus 0.01% BP, and 0.01% AI (Groups 4 to 8), respectively, had significantly less lesion scores than the infected unmedicated birds (Group 2). The data suggest that a combination of AI and BP reduced gross lesions in chicken guts more than either one alone.

TABLE 4

| | Gross lesion score | | | | | | | |
|---|---|---|---|---|---|---|---|---|
| Group | 0 | 1 | 2 | 3 | 4 | Average | P value^{a} | P value^{b} |
| 1 (n = 15, 0) | 14/15 | 0/15 | 0/15 | 0/15 | 0/15 | 0 ± 0 | | |
| 2 (n = 15, 1 × 10⁴) | 0/15 | 0/15 | 0/15 | 0/15 | 10/15 | 4.00 ± 0.00 | <0.0001 | |
| 3 (n = 15, 1 × 10⁴) | 0/15 | 0/15 | 0/15 | 1/15 | 6/15 | 3.86 ± 0.10 | <0.0001 | 0.208 |
| 4 (n = 15, 1 × 10⁴) | 0/15 | 1/15 | 3/15 | 4/15 | 1/15 | 2.56 ± 0.23 | <0.0001 | <0.0001 |
| 5 (n = 15, 1 × 10⁴) | 0/15 | 5/15 | 3/15 | 4/15 | 0/15 | 2.14 ± 0.28 | <0.0001 | <0.0001 |
| 6 (n = 15, 1 × 10⁴) | 0/15 | 5/15 | 3/15 | 4/15 | 0/15 | 1.92 ± 0.23 | <0.0001 | <0.0001 |
| 7 (n = 15, 1 × 10⁴) | 0/15 | 6/15 | 1/15 | 2/15 | 0/15 | 1.56 ± 0.23 | <0.0001 | <0.0001 |
| 8 (n = 15, 1 × 10⁴) | 0/15 | 0/15 | 5/15 | 1/15 | 4/15 | 2.90 ± 0.26 | <0.0001 | <0.05 |

The numerator and denominator in a fraction represent the number of chickens with cecal gross lesions in 5 grading categories (0 to 4). The number (n) represents the total number of chickens in each group. The difference in the gross lesions of the ceca of chickens between the infected medicated groups and infected unmedicated group (Group 2) is analyzed by chi-square test, after multinomial transformation and shown by P value.

Table 5 shows microscopic lesion scores in the ceca of the chickens from FIG. 4B on Day 7 post ET infection. Mucosal damages caused by coccidia were examined under microscope and scored as microscopic cecal lesions based on the distribution and severity of the mucosal destructions in the chicken cecum.

No microscopic cecal lesions (score=0) were observed in the uninfected unmedicated control group (Group 1). The infected unmedicated animals (Group 2) showed serious microscopic lesions (score=7.2) in the cecum 7 days after the ET infection. Severe ulceration, hemorrhage and decreased villi in the cecum were also observed (data not shown). Oocysts, gametocytes and schizonts appeared inside the cecal epithelia (data not shown). The infected salinomycin-fed animals (Group 3) showed mild improvement in microscopic lesions (score=6.29) in the cecum as compared to the infected unmedicated animals (Group 2) post infection. However, the infected animals fed with B. pilosa, B. pilosa combining AI, or AI alone (Groups 4 to 8, Table 5) showed significantly reduced microscopic lesions (scores of 4.78 to 5.80) in the cecum. Consistently, B. pilosa decreased ulceration and hemorrhage and preserved more mucosae and villi in chicken ceca than control diets (data not shown). B. pilosa also decreased the number of oocysts, gametocytes and schizonts inside the cecal epithelia to a greater extent than salinomycin and control diets (data not shown). Overall, B. pilosa, combination of B. pilosa and AI, or AI alone significantly reduced gut pathology in chickens following ET infection. The data suggest that a combination of AI and BP reduced microscopic lesions in chicken guts more than either one alone.

TABLE 5

| | Microscopic lesion score¹ | | | | | | P value² | |
|---|---|---|---|---|---|---|---|---|
| Group | 0 | 1 | 3 | 5 | 7 | Average | P^{a} | P^{b} |
| 1 (n = 15, 0) | 0/15 | 0/15 | 0/15 | 0/15 | 0/15 | 0 ± 0 | <0.0001 | |
| 2 (n = 15, 1 × 10⁴) | 0/15 | 0/15 | 0/15 | 0/15 | 4/15 | 7.20 ± 0.20 | <0.0001 | |
| 3 (n = 15, 1 × 10⁴) | 0/15 | 0/15 | 0/15 | 0/15 | 2/15 | 6.29 ± 0.13 | <0.0001 | <0.05 |
| 4 (n = 15, 1 × 10⁴) | 0 15 | 0/15 | 0/15 | 4/15 | 1/15 | 4.78 ± 0.25 | <0.0001 | <0.0001 |
| 5 (n = 15, 1 × 10⁴) | 0/15 | 0/15 | 0/15 | 4/15 | 0/15 | 4.57 ± 0.14 | <0.0001 | <0.0001 |
| 6 (n = 15, 1 × 10⁴) | 0/15 | 0/15 | 2/15 | 2/15 | 0/15 | 3.83 ± 0.22 | <0.0001 | <0.0001 |
| 7 (n = 15, 1 × 10⁴) | 0/15 | 0/15 | 2/15 | 4/15 | 0/15 | 3.44 ± 0.26 | <0.0001 | <0.0001 |
| 8 (n = 15, 1 × 10⁴) | 0/15 | 0/15 | 0/15 | 0/15 | 3/15 | 5.80 ± 0.27 | <0.0001 | <0.05 |

¹Microscopic lesion score is represented as a fraction, in which the numerator and denominator are indicated. The numerator and denominator in a fraction represent the sum of microscopic lesions (0 to 4) in the gut samples, 5 section slides per gut, and the number of the examined gut samples multiplied by 5 per group.
²The difference in microscopic lesion scores of the chickens between infected medicated groups and uninfected unmedicated group (Group 1) is analyzed with a chi-square test after multinomial transformation and shown by P^{a} value. Similarly, the difference in microscopic lesion scores of the chickens between infected medicated groups and infected unmedicated group (Group 2) is shown by P^{b} value.

Effect of Plant Extracts on Bloody Stools in E. tenella Challenged Chickens

FIG. 5 shows percentage of bloody stools in each chicken group on Day 7 post E. tenella (ET) infection. The uninfected control chickens (Group 1) had no bloody stools. The ET infection caused 100% of bloody stools (Group 2). The infected birds fed with salinomycin showed a significantly decrease in the percentage of bloody stools (Group 3). The infected chickens fed with 0.01% BP, 0.0001% AI plus 0.01% BP, 0.001% AI plus 0.01% BP, 0.01% AI plus 0.01% BP, and 0.01% AI (Groups 4 to 8), respectively, showed significantly decrease in the percentage of bloody stools than the infected unmedicated birds (Group 2). The data suggest that a combination of AI and BP reduced more bloody stools than either one alone.

All references cited and discussed in this specification are incorporated herein by reference in their entireties and to the same extent as if each reference was individually incorporated by reference.

1. An anti-coccidial composition comprising:
(a) an effective amount of Artemisia indica extract;
(b) an effective amount of Bidens pilosa extract, and
(c) a pharmaceutically acceptable vehicle, excipient, or carrier.

2. The anti-coccidial composition of claim 1, which is an oral dosage form.

3. The anti-coccidial composition of claim 2, wherein the oral dosage form is selected from the group consisting of tablets, pills, softgel capsules, hard capsules, granules, powders, concentrates, liquids, molded balls and any combinations thereof.

4. The anti-coccidial composition of claim 1, further comprising an animal feed.

5. The anti-coccidial composition of claim 4, wherein the animal feed is selected from the group consisting of poultry feed, fish food, amphibian feed, reptile food, bird feed, and non-human mammal food.

6. The anti-coccidial composition of claim 1, wherein the Artemisia indica extract and the Bidens pilosa extract are present in a ratio of about from 0.0001:1.0 to 1.0:1.0.

7. The anti-coccidial composition of claim 1, wherein the amount of Artemisia indica extract ranges from 0.0001% to 0.01% by weight of the composition.

8. The anti-coccidial composition of claim 6, wherein the amount of Bidens pilosa extract ranges from 0.01% to 0.1% by weight of the composition.

9. The anti-coccidial composition of claim 1, wherein the Artemisia indica extract comprises chlorogenic acid.

10. An anti-coccidial composition comprising:
(a) an effective amount of Artemisia indica extract; and
(b) an effective amount of Bidens pilosa extract.

11. The anti-coccidial composition of claim 10, which is in a non-aqueous liquid, aqueous liquid, suspension or powder form.

12. A method for killing coccidian oocysts and inhibiting coccidian oocyst sporulation, comprising administering to a subject in need thereof the anti-coccidial composition of claim 10.

13. A method for killing coccidian oocysts, inhibiting oocyst sporulation, reducing sporozoite invasion, and lessening bloody stools in a subject in need thereof, comprising administering to the subject in need thereof the anti-coccidial composition of claim 1.

14. A method for alleviating or treating coccidiosis in a subject in need thereof, comprising administering to the subject in need thereof the anti-coccidial composition of claim 4.

15. (canceled)

16. An anti-coccidial composition consisting essentially of:
(a) an effective amount of Artemisia indica extract;
(b) an effective amount of Bidens pilosa extract, and
(c) a non-human animal feed or food.

17. The anti-coccidial composition of claim 4, wherein the Artemisia indica extract and the Bidens pilosa extract are present in a ratio of about from 0.0001:1.0 to 1.0:1.0.

18. The anti-coccidial composition of claim 17, wherein the amount of Bidens pilosa extract ranges from 0.01% to 0.1% by weight of the composition.

19. The anti-coccidial composition of claim 4, wherein the amount of Artenisia indica extract ranges from 0.0001% to 0.01% by weight of the composition.

20. The anti-coccidial composition of claim 19, wherein the amount of Bidens pilosa extract ranges from 0.01% to 0.1% by weight of the composition.

21. A method for killing coccidian oocysts, inhibiting oocyst sporulation, reducing sporozoite invasion, and lessening bloody stools in a subject in need thereof, comprising administering to the subject in need thereof the anti-coccidial composition of claim 10.
